(19) United States
(12) Patent Application Publication
(10) Pub. No.: US 2005/0228466 A1
(43) Pub. Date: Oct. 13, 2005

(54) Infant heel warmer

(76) Inventor: Christa Harris, Waukegan, IL (US)

Correspondence Address: ALLEGIANCE CORPORATION;Attn: Kim Luna, KB-1A, 1430 Waukegan Rd, McGaw Park, IL, 60085-6787, US

(21) Appl. No.: 10/821,694

(22) Filed: Apr. 9, 2004

(51) Int. Cl. A61F 7/00

(52) U.S. Cl. 607/114

(57) ABSTRACT

The invention described herein is an improved infant heel warming device containing an exothermic thermochemical composition that can be activated at the time of use to provide a warming effect to the skin. The infant heel warming device comprises a flexible polymeric containment for said thermochemical composition, the containment being structured to have a first skin-contacting side and second viewing side. The first skin-contacting side further comprises an outer fabric layer attached thereto. The second viewing side is composed of a transparent polymeric material permitting viewing of the interior of the flexible polymeric containment and its contents. The device further comprises at least one securing structure attached to the device to maintain the position of the device onto an infant's foot. The device enhances comfort to an infant's sensitive skin through the use of a soft, friction-enhancing insulation barrier and affords readily apparent viewing of the interior contents.

### FIELD OF THE INVENTION

The invention relates to the field of thermal medical devices. In particular, the invention pertains to an improved thermal device for infants.

### BACKGROUND OF THE INVENTION

Thermal devices, specifically thermochemically activated thermal device, have been available in a wide variety of structures, configurations and for various uses and applications. In the medical field, thermochemically activated thermal devices, such as thermal packs, have been designed to conform to various patient geometries in order to deliver therapeutic thermal treatments using heat or cooling applications.

One family of such devices are known as infant heel warmers. Infant heel warmers are used to perform a specific medical preparatory function, e.g., warming an infant's heel prior to drawing a blood sample therefrom. Infant heel warmers, in addition to conformational geometry, have also been constructed with straps or other securing means to facilitate the maintenance of the heel warmer on the infant's foot. Examples of such infant heel warmers include those described in Manker U.S. Pat. No. 5,800,492.

One problem associated with using exterior pouches with thermal packs placed therein is that the insulation material completely encases the thermal pack and prevents the user from viewing the pack. Another problem associated with conventional thermal packs is the use of opaque flexible films, which also obscures viewing or observation of the internal chemical component therein. While thermal packs can be entirely constructed of transparent film, the interior chemical component cannot be viewed and inspected by an individual, and the advantages of having insulation interposed between the film exterior and the user's skin are absent.

Another problem associated with such strapped heel warmers is that even though fitted like a “sandal”, the kicking movements exerted by the infant often results in the migration of the device on the infant's foot. While adjusting the straps tighter around the foot would indeed better secure the device, it would also produce the undesirable consequence to blood circulation in the foot.

It would be desirable to have an infant heel warmer that provides comfortable and safe thermal attributes to the infant while affording the individual-practitioner readily observable usage features. Even more desirable would be a thermal device that accommodates the importance of insulation during warming therapy against an infant's sensitive skin without compromising the advantages of individual viewing capability. Thus, there exists a need in the field of thermal medical devices for improved infant heel warming devices that address these concerns.

### SUMMARY OF THE INVENTION

The invention provides an improved thermal device in the form of an infant heel warming device containing a thermochemical composition within that is activated at the time of use to provide an exothermic reaction. It has been discovered that an infant heel warming device can be constructed so as to reduce the likelihood of undesired slippage and migration of the device on the infant's foot without undue constriction from straps, provide comfort to an infant's sensitive skin through the use of a soft, friction-enhancing insulation barrier, and provide readily apparent viewing of the chemical contents by the practitioner or other individual—all without compromising the structural and functional attributes associated with an infant heel warmer.

In particular, the invention provides an infant heel warming device comprising:
an exothermic thermochemical composition wherein the exothermic reaction occurs upon activation;
a flexible polymeric containment for the thermochemical composition, said containment being structured to have a first skin-contacting side and second viewing side, each of the first and second sides having an interior surface and exterior surface;
wherein said first skin-contacting side further comprises an outer fabric layer attached thereto.

The second viewing side of the containment is composed of a transparent polymeric material permitting viewing of the interior of said flexible polymeric containment. The device further comprises at least one securing strap attached to the device to secure the device onto an infant's foot.

In a preferred embodiment, the exothermic thermochemical composition of the infant heel warming device comprises a liquid or fluid composition together with a physical activator within the fluid.

One advantage of the invention is that the skin-contacting fabric layer of the first side of the device provides both an insulating barrier to reduce tactile and thermal discomfort to the skin. Furthermore, the fabric provides a friction enhancing surface to the device to reduce the likelihood of undesired slippage and migration of the device as a result of the infant's motion during use. Another advantage of the invention is that the second viewable side of the device permits the individual to locate the physical activator to initiate the exothermic reaction, as well as permit the individual to continually observe the interior of the containment throughout the duration of the use of the device.

Other aspects of the invention and advantages will become apparent from the following drawings and description.

### BRIEF DESCRIPTION OF THE DRAWINGS

The invention is further illustrated by the following figures, none of which are to be construed as necessarily limiting the invention:

FIG. 1 is an overall view of the infant heel warming device according to one embodiment of the invention.

FIG. 2 is a view of the device from the skin contacting side according to one embodiment of the invention.

FIG. 3 is a view of the opposing side of that depicted in FIG. 2 showing the transparent side of the device according to one embodiment of the invention.

FIG. 4 is a side view of the device according to one embodiment of the invention.

FIG. 5 is a cross-sectional side view from one end of the device showing the device construction according to one embodiment of the invention.

FIG. 6 is a cut-away angled side view of the device showing the interior layers and contents of the device according to one embodiment of the invention.

FIG. 7 is an illustration of the device of the invention positioned and secured onto an infant's foot according to one embodiment of the invention.

### DETAILED DESCRIPTION OF THE INVENTION

As used herein, the term “transparent”, when used in the context of material, refers to the property of permitting viewing of contents beyond the opposing side of such material in a substantially clear manner. The term is meant to include colored transparent materials.

As used herein, the term “trigger” when used in the context of a crystallization activator is meant to refer to the generally planar and flexible devices that, upon flexing within thermochemical fluid, initiate crystallization and the subsequent exothermic effect. Such devices are described, for example, in U.S. Pat. Nos. 4,460,546, 4,572,158, 4,872,442, 5,143,048, 5,736,110, and 6,283,116, the entire texts of which are incorporated herein by reference.

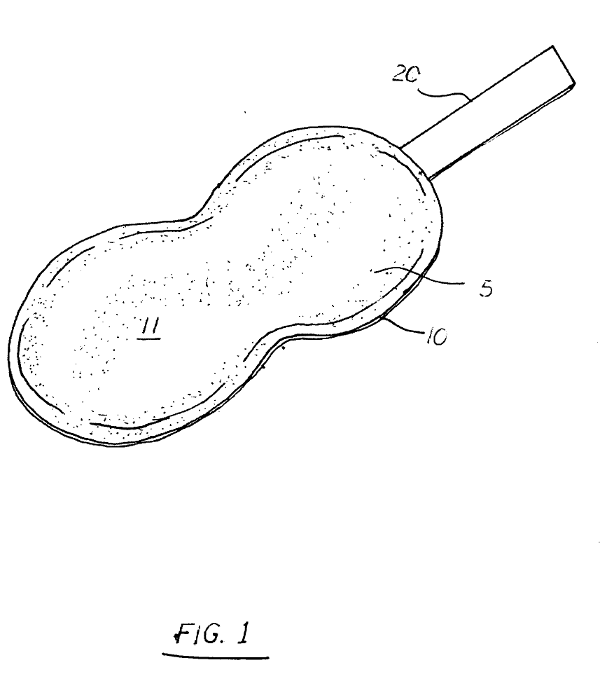

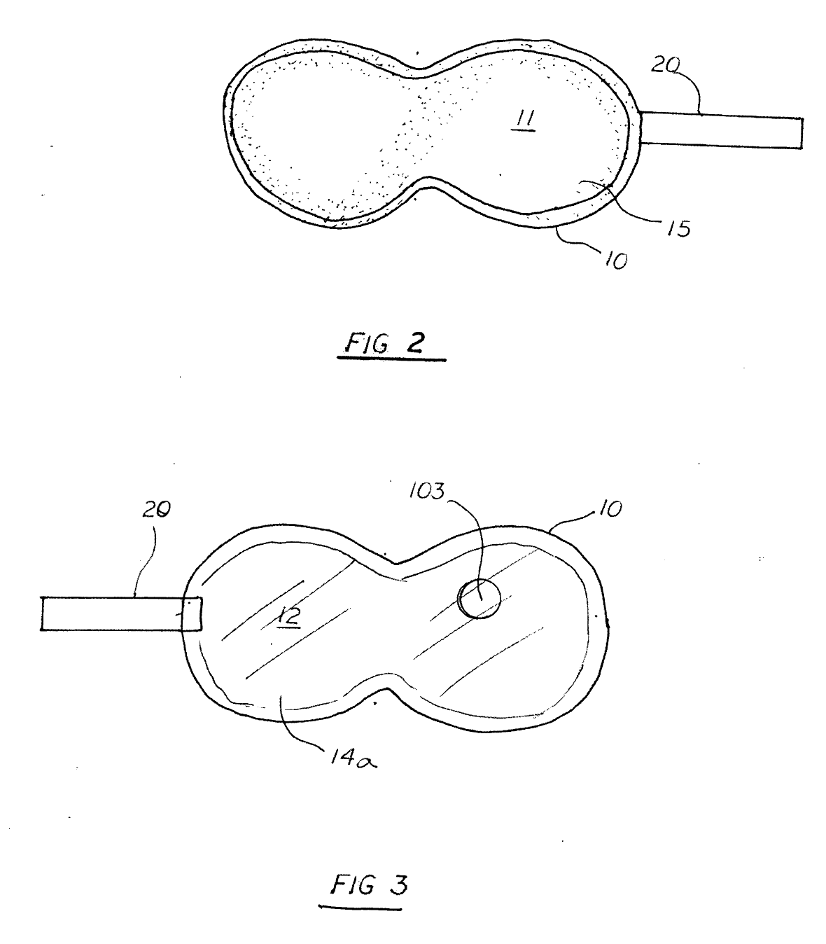

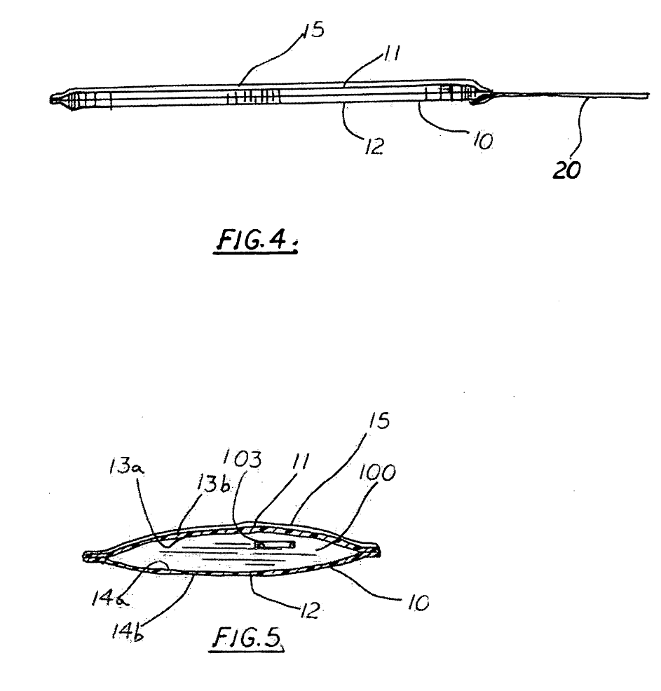

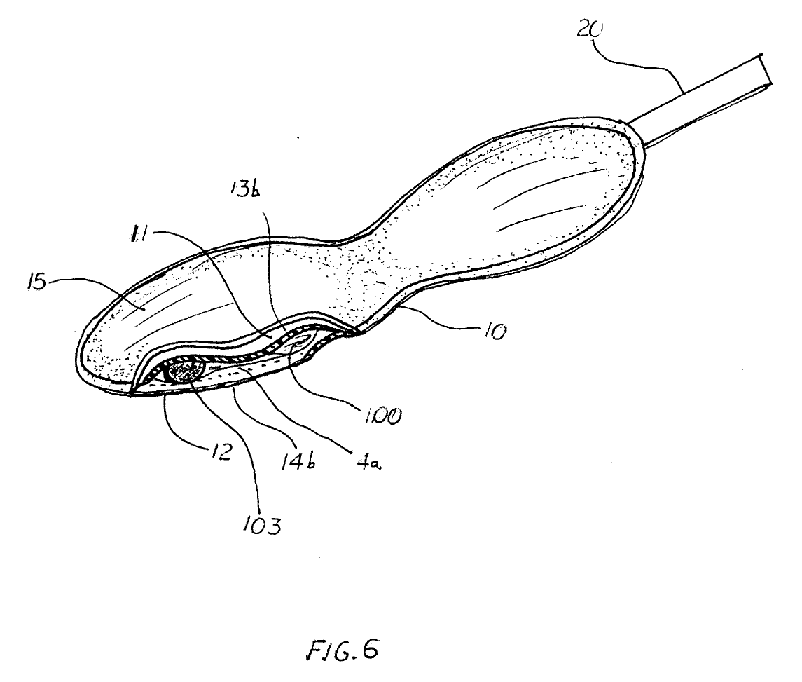

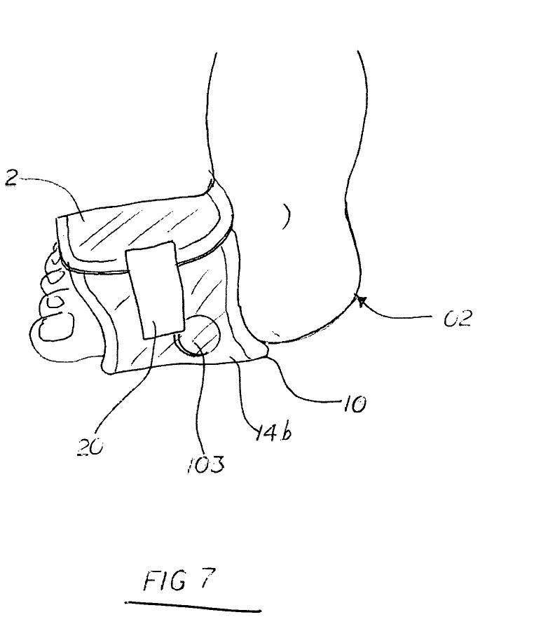

In general, the infant heel warming device of the invention, by virtue of its improved instruction in accordance with the invention, affords advantages to both the infant user and the individual applying the device to the heel. Referring to FIG. 1, the device of the invention comprises an exothermic thermochemical composition 100 (see FIGS. 5 and 6) within a liquid-proof flexible polymeric containment 10. An exothermic chemical reaction occurs upon activation of the reaction at the time of use. Now referring to FIGS. 3, 5, 6 and 7, the flexible polymeric containment 10 can be structured to have a first skin-contacting side 11 and second viewing side 12, each of the first and second sides having an interior surface 13a and 14a respectively, and exterior surface 13b and 14b respectively. The first skin-contacting side 11 of the device further comprises a fabric layer 15 attached thereto. The second viewing side 12 can be composed of a transparent polymeric material permitting viewing of the interior of said flexible polymeric containment 10 as illustrated in FIGS. 3 and 7. The device further comprises at least one securing structure attached to, or associated with, the device to secure the device onto an infant's foot.

A variety of thermochemical compositions which are activatable at time of use are available and can be used in accordance with the invention. Suitable thermochemical compositions that can be used include those wherein two or more chemical components are physically separated by a barrier. Such compositions activate by virtue of the combination of separate chemical components, wherein the physical barriers separating the components are ruptured. Examples of this type of exothermic composition can include, but are not limited to: sodium thiosulfate liquid and borax solid; sodium acetate liquid and sodium acetate solid; magnesium sulfate compositions; and the like.

In a preferred embodiment, the device of the invention contains a single liquid thermochemical composition that produces exothermic temperatures alongside its crystallization, and a physical activator 103 (illustrated as a disc in FIGS. 3, 5, 6 and 7) associated within the composition. Physical activators 103 can be can be readily and visibly located by the individual and then activated at time of use. This category of activator has been referred to in a variety of ways in the art, for example as “clickers” and initiators, and are herein referred to as triggers. By virtue of their structure, the trigger can be a compact, flat, relatively small structure, the rubbing, bending or flexing of which initiates crystallization of the thermochemical composition. The crystallization in turn is associated with exothermic warming temperatures. The use of a trigger facilitates activation of the thermochemical composition of the device.

Physical activators that can be used in accordance with the invention can include triggers in the form of particles adhered to a substrate surface or flexible metallic discs. Examples of such triggers include, but are not limited to, aluminum oxide particle or grit surface materials and flexible stainless steel triggers. Flexible stainless steel triggers can comprise a flat stainless steel disc containing one or more slots or openings therethrough.

For exothermic thermochemical liquid composition and physical activator systems, suitable exothermic thermochemical liquids include, but are not limited to, sodium acetate trihydrate. In a preferred embodiment, the thermochemical composition comprises a mixture of sodium acetate trihydrate present in an amount of about 73% of the total liquid volume and water present in an amount of about 27% of the total liquid volume (available from Niacet, Niagara Falls, N.Y.); and the physical activator comprises a trigger comprising an aluminum oxide grit (available from Abrasive Specialties, Cincinnati, Ohio) or comprising a flexible stainless steel trigger.

The device of the invention comprises a flexible polymeric containment 10 for the thermochemical composition 100, wherein the containment 10 is conformable to the infant patient's foot geometry. The containment 10 can have an overall flat or planar configuration. A variety of containment shapes and configurations can be used in accordance with the invention. In one embodiment and as shown in the figures, the containment 10 comprises a relatively flat hourglass or peanut contoured shape. When this configuration is employed, the foot 102 is placed across the first skin-contacting side of the device containing the fabric layer, so that the longitudinal axis of the foot is across the narrow region of the device (see FIG. 7).

The flexible polymeric containment can be constructed from a variety of flexible polymeric film. Suitable film materials that can be used include polyester, polypropylene, polyethylene, nylon, ethyl vinyl acetate (EVA), and combinations thereof. The flexible polymeric containment material can be single layered or multilayered. A preferred flexible polymeric film material for the invention is a thermal barrier film as described in co-pending U.S. patent application Ser. No. ______ (not yet assigned), filed on ______ (not yet determined), the content of which is incorporated herein by reference. This thermal barrier film comprises an interior layer composed of a blend of linear low density polyethylene (LDPE) and ethyl vinyl acetate (EVA), and polyester layer coated with aluminum oxide.

The first skin-contacting side 11 of the device further comprises an outer fabric layer 15 attached to the surface of the polymeric film. A variety of fabric materials can be used, including but not limited to, woven and non-woven fabrics. Preferred fabric materials for use with the invention are those that are comfortable and soft to the touch, while at the same time permitting warming temperature penetration therethrough. In a preferred embodiment, the fabric is a non-woven. Suitable non-woven fabric materials include, but are not limited to, polyester, polyethylene, polypropylene, rayon, and the like. The non-woven fabric materials can be spunbond, meltblown, or spunlace. A preferred fabric materials for use with the device of the invention is the polyester fabric MIRATEC® (available from PGI Polymeric Group, Inc., Mooresville, N.C.).

The fabric layer can be attached to the first skin-contacting side of the containment using a variety of conventional techniques readily available to those skilled in the art. For example, the fabric layer can be laminated, adhered, or point bonded onto the film layer of the surface. The fabric layer can cover the entire surface of the skin-contacting side as shown in the figures. Alternatively, the fabric layer can partially cover the skin-contacting side, provided the advantageous insulating and friction-enhancing properties associated with the device are not substantially compromised.

The second viewing side 12 of the device can be composed of a transparent polymeric film material thereby permitting viewing of the interior of the containment. This permits the individual to readily locate the physical activator 103 within the liquid thermochemical composition 100 as illustrated in FIGS. 3 and 7.

Although both polymeric film layers which form the containment of the device of the invention can be transparent, it is not necessary that the film layer upon which the fabric layer is fixed be transparent as well. The advantages associated with second side transparency of the invention can be fully appreciated from the second side transparency alone.

The device of the invention further comprises at least one securing structure attached to the device to secure said device onto an infant's foot (see for example FIG. 7). A variety of securing structures and materials can be used, provided they can secure the placement and positioning of the thermal device containment onto the user's foot geometry. One securing structure that can be used in accordance with the invention is a securing strap 20 as illustrated. The securing strap can be elastic or inelastic. In one embodiment, the securing strap 20 can be composed of a flexible plastic strip coated on one side with an adhesive. For example, the securing strap 20 can be constructed as a non-woven strip containing an acrylic copolymer adhesive, and a removable kraft paper with a silicone release coating on the surface contacting the adhesive. Alternatively, the securing strap can be a hook and loop fastener, a plastic strip coated on one surface with a low-tack adhesive, and the like. In yet another embodiment, the securing structure can be in the form of an adhesive pad or region on the exterior of the device that adheres or tacks onto the second surface without the use of a strap.

An important aspect of the invention is that the maintenance of the desired placement on the infant user's foot is not solely reliant upon the strap. The fabric surface of the first skin-contacting side of the containment affords some frictional resistance to the tendency for such devices to migrate or slip. This movement is often caused by repositioning the infant or the infant's own kicking and movements.

The infant heel warming device of the invention can be manufactured using conventional equipment and techniques readily available to those skilled in the art. For example, the containment can be constructed as a thermal sealed bag structure, or two sheets with perimeters sealed. Generally, the heel warmer device of then invention can be assembled by heat sealing layers of the flexible polymeric film, one laminated on the outer surface with a non-woven material, around a predetermined liquid fill volume content. A securing strap can then be manually applied onto the device.

In use, the infant's foot is prepared to receive the infant heel warmer device, and the device is thermochemically activated by an individual. In the case of a device comprising a trigger-type physical activator, the trigger can be located and grasped by the individual through the outside of the containment. Once the trigger is grasped, the containment material and trigger adjacent thereto can be bent, rubbed, or flexed until the exothermic reaction is initiated and proceeds. Initiation of the exothermic reaction can be confirmed by touching the device on the second viewing side (without the intervening fabric layer), or by simply viewing the commencement of crystallization with certain exothermic liquid compositions. The device of the invention affords the advantage of such a rapid determination. Alternatively, the device can be secured partially or completely onto the infant's foot and then activated thusly.

With the warming effect confirmed, the individual can then position the first skin-contacting side 11 of the device adjacent the skin of the infant's foot 102 with the second viewable side 12 facing outward as shown in FIG. 7 so as to permit viewing of the interior compartment through the transparent film. The securing structure 20 can be fastened, thereby securing the device onto the foot 102. An important aspect of the invention is that the outer or exterior fabric layer 15 on the first skin-contacting side 11 of the device enhances the comfort of the device and functions as an insulating barrier to prevent or reduce the likelihood of injury or burns to the infant's sensitive skin during use. Yet another important aspect of the outer fabric layer 15 is that friction can be maintained without sacrificing the use of protective insulation and temperature buffering against the skin. In a further embodiment, additional or secondary friction-enhancing materials or texturing can be applied partially or completely to the outer fabric layer 15.

### INDUSTRIAL APPLICABILITY

The invention is useful to provide a thermal warming effect to an infant's foot. The invention is particularly useful in the medical environment, wherein warming of the foot is desired in advance of a procedure by which blood is drawn from an infant's foot.

The invention has been described herein above with reference to various and specific embodiments and techniques. It will be understood, however, that reasonable modifications and variations of such embodiments and techniques can be made without significantly departing from either the spirit or scope of the invention defined by the following claims.

1. A warming device comprising:
an exothermic thermochemical composition wherein the exothermic reaction occurs upon activation;
a flexible polymeric containment for said thermochemical composition, said containment being structured to have a first skin-contacting side and second transparent viewing side, each of said first and second sides having an interior surface and exterior surface;
wherein said first skin-contacting side of said containment further comprises an outer fabric layer attached onto said skin-contacting side surface.

2. The device according to claim 1, wherein the warming device is an infant heel warming device.

3. The device according to claim 1, wherein wherein said second viewing side is composed of a transparent polymeric material permitting viewing of the interior of said flexible polymeric containment.

4. The device according to claim 1, wherein at least one securing strap is attached to said device to secure said device onto the foot.

5. The device according to claim 1, wherein said outer fabric layer is laminated onto said first skin-contacting side.

6. The device according to claim 1, wherein said outer fabric layer is adhered onto said first skin-contacting side by an adhesive.

7. The device according to claim 1, wherein said outer fabric layer is composed of a non-woven material.

8. The device according to claim 7, wherein said non-woven material is selected from the group consisting essentially of polyester, polyethylene, polypropylene, and rayon, and combinations thereof.

9. The device according to claim 1, wherein said thermochemical composition comprises a liquid mixture of sodium acetate trihydrate and water.

10. The device according to claim 9, wherein the thermochemical composition comprises a liquid mixture of sodium acetate trihydrate in an amount of about 73% of the total liquid volume and water in an amount of about 37% of the total liquid volume.

11. The device according to claim 1, further comprising a physical activator.

12. The device according to claim 11, wherein the physical activator comprises particles adhered to a substrate surface.

13. The device according to claim 12, wherein said particles comprise aluminum oxide grit.

14. The device according to claim 11, wherein the physical activator is a flexible metallic disc.

15. The device according to claim 14, wherein the flexible metallic disc is composed of stainless steel.
